(19) United States
(12) Patent Application Publication
(10) Pub. No.: US 2005/0232885 A1
(43) Pub. Date: Oct. 20, 2005

(54) Detergent cosmetic compositions comprising at least one surfactant, at least one drawing polymer and at least one nonsilicone conditioner, and use thereof

(75) Inventors: Henri Samain, Bievres (FR); Geraldine Fack, Levallois (FR)

Correspondence Address: Thomas L. Irving;FINNEGAN, HENDERSON, FARABOW,, GARRETT & DUNNER, L.L.P., 1300 I Street, N.W., Washington, DC, 20005-3315, US

(73) Assignee: L'OREAL

(21) Appl. No.: 11/030,418

(22) Filed: Jan. 7, 2005

Related U.S. Application Data
(60) Provisional application No. 60/537,555, filed on Jan. 21, 2004.
(60) Provisional application No. 60/537,902, filed on Jan. 22, 2004.

(30) Foreign Application Priority Data
Jan. 7, 2004 (FR) 0450026
Jan. 7, 2004 (FR) 0450032

(51) Int. Cl. A61K 7/06; A61K 7/11

(52) U.S. Cl. 424/70.11

(57) ABSTRACT

The present disclosure relates to novel washing compositions, such as hair compositions, comprising, in a cosmetically acceptable medium, at least one water-insoluble nonsilicone conditioner, at least one detergent surfactant chosen from anionic, nonionic and amphoteric detergent surfactants, and at least one drawing polymer. The present disclosure further relates to a process for treating hair using the compositions disclosed herein.

This application claims benefit of U.S. Provisional Application No. 60/537,555, filed Jan. 21, 2004, and U.S. Provisional Application No. 60/537,902, filed Jan. 22, 2004.

The present disclosure relates to novel cosmetic compositions with improved properties, intended for cleaning, conditioning and styling keratin materials such as the hair, and comprising, in a cosmetically acceptable support, a washing base comprising at least one surfactant with detergent power, in which at least one water-insoluble nonsilicone conditioner is also present in combination with drawing polymers. The present disclosure also relates to a process for cleaning and/or conditioning and/or styling keratin materials comprising the application of the compositions as disclosed herein.

It is common practice to use detergent hair compositions (or shampoos) based essentially on standard surfactants of anionic, nonionic and/or amphoteric type, for example, such as of anionic type, to clean and/or wash the hair. These compositions are applied to wet hair and the lather generated by massaging or rubbing with the hands can remove, after rinsing with water, the various types of soiling initially present on the hair.

Although these base compositions can have good washing power, the intrinsic cosmetic properties associated therewith can nevertheless remain fairly poor, owing, for example, to the fact that the relatively aggressive nature of such a cleaning treatment can, in the long run, lead to more or less pronounced damage to the hair fiber. This damage can be associated, for instance, with the gradual removal of the lipids or proteins contained in or on the surface of this fiber.

Thus, in order to improve the cosmetic properties of the above detergent compositions, such as those that are intended to be applied to sensitized hair (i.e., hair that has been damaged or made brittle, for instance, due to the chemical action of atmospheric agents and/or hair treatments such as permanent-waving, dyeing or bleaching), it is now known to introduce additional cosmetic agents known as conditioners into these compositions, these conditioners being intended mainly to repair or limit the harmful or undesirable effects induced by the various treatments or aggressions to which the hair fibers can be subjected more or less repeatedly. These conditioners may, of course, also improve the cosmetic behavior of natural hair.

The conditioners most commonly used to date in shampoos are cationic polymers, which can give washed, dry or wet hair at least one cosmetic property chosen from an ease of disentangling, softness, and smoothness that can be markedly better than those that may be obtained with corresponding cleaning compositions from which cationic polymers are absent.

Moreover, it has for some time been sought to obtain conditioning shampoos that are capable of giving washed hair not only the cosmetic properties mentioned above but also, to a greater or lesser extent, styling, volume, shaping and hold properties. Such washing shampoos with improved general cosmetic properties are often referred to for simplicity as “styling shampoos,” and this term will be adopted in the description hereinbelow.

However, despite the progress made recently in the field of styling shampoos, these shampoos are not always satisfactory, and as such there is currently still a strong need to be able to provide novel products that give better performance with respect to at least one of the cosmetic properties mentioned above. For example, it is usually necessary to use a styling product after shampooing, to give the hair a shape and to fix the style. The present disclosure is directed towards satisfying such a need.

Thus, the present inventors have now found, surprisingly and unexpectedly, that by combining at least one water-insoluble conditioner, as defined below, and at least one anionic, nonionic, zwitterionic or amphoteric detergent surfactant, with at least one polymer that has a particular drawing power in detergent compositions, it is possible to substantially and significantly improve the styling and hold properties, while at the same time maintaining their good intrinsic washing power and their cosmetic properties.

For example, these compositions can make it possible to obtain very good hold and/or a certain amount of volume for the hair, i.e., a styling effect similar to that obtained with a fixing styling gel used after shampooing. It moreover may be found that the keratin fibers are strengthened.

All these discoveries form the basis of the present disclosure.

Accordingly, the present disclosure relates to novel compositions for washing keratin materials, such as the hair, comprising, in a cosmetically acceptable aqueous medium, at least one water-insoluble nonsilicone conditioner, at least one detergent surfactant chosen from anionic, nonionic, zwitterionic and amphoteric detergent surfactants, and at least one polymer with a drawing power of greater than 5 cm.

One aspect of the present disclosure is also the cosmetic use of the above compositions for cleaning, conditioning, caring for and styling keratin materials, for instance, the hair and the eyelashes.

Another aspect of the present disclosure relates to a cosmetic process for treating keratin materials, such as keratin fibers, e.g., the hair and the eyelashes, using the compositions as disclosed herein.

Still another aspect of the present disclosure is the use of the composition as disclosed herein as a shampoo.

However, other characteristics, aspects and benefits of the present invention will emerge even more clearly upon reading the description that follows, and also the concrete, but in no way limiting, examples intended to illustrate it.

As used herein, the term “drawing power of a polymer” is understood to mean the length of the polymer yarn obtained at the breaking point of the yarn according to the procedure defined below.

The drawing power of the polymers that may be used as disclosed herein is the power measured for a composition comprising (% by weight):

| | |
|---|---|
| Sodium lauryl ether sulphate oxyethylenated | 12.5% |
| with 2.2 mol of ethylene oxide | |
| Cocoamidopropylbetaine | 2.5% |
| Test polymer | 1% |
| Water qs | 100% |

The drawing power is measured using a TA-TX2 texture analyzer (Rheo/stable Micro Systems).

The measurement is performed after compression of the product:
Displacement of the disc (35 mm aluminium cylinder) at a speed of 2.5 mm/s and detection of the compression strength
penetration into the product at the same speed to a depth of 10 mm
removal of the probe at a speed of 2.5 mm/s
measurement of the displacement of the probe and detection of the breaking point of the product yarn.

In the present disclosure, the polymers with a drawing power of greater than 5 cm will also be referred to as “drawing polymers.”

The polymers with a drawing power of greater than 5 cm can be chosen from, for example, either (a1) a dispersion of particles of at least one water-soluble cationic polymer with a weight-average molecular mass of greater than 10⁶ (also referred to below as a “high molecular weight water-soluble cationic polymer”) in a saline aqueous solution, obtained by heterogeneous free-radical polymerization of water-soluble monomers with precipitation of the polymer formed, or (a2) an aqueous saline solution of at least one water-soluble cationic polymer with a weight-average molecular mass of greater than 10⁶, obtained by heterogeneous free-radical polymerization of water-soluble monomers with precipitation of the polymer formed.

As used herein, the term “cationic polymer” includes polymers comprising cationic monomers and possibly nonionic monomers.

The high molecular weight water-soluble cationic polymer can be a polyelectrolyte polymerized using at least one cationic monomer of formula (I) as described below.

As disclosed above, the synthesis of the high molecular weight water-soluble cationic polymers used herein takes place by heterogeneous free-radical polymerization of water-soluble monomers comprising at least one ethylenic unsaturation. The polymerization takes place in an aqueous solution of a mineral electrolyte (salt) having an ionic strength that is sufficient to cause precipitation of the polymer formed as soon as the polymer has reached a certain molecular mass. This polymerization technique thus allows, by virtue of the well-known phenomenon of salting out, the preparation of saline aqueous dispersions of water-soluble polymer particles. The polymers thus synthesized are distinguished by a high weight-average molecular mass, which is generally greater than 10⁶.

The technique of heterogeneous free-radical polymerization in an aqueous medium with precipitation of the polymer formed is described, for example, in U.S. Pat. No. 4,929,655, in European Patent Application No. EP 0 943 628 or in International Patent Application No. WO 02/34796.

To ensure the stability of the dispersions of polymer particles during the synthesis and during storage, it is helpful to perform the polymerization in the presence of a dispersant. This dispersant can be, for example, a polyelectrolyte, which, unlike the high molecular weight water soluble cationic polymer described above, is soluble in the aqueous polymerization medium of high ionic strength. This dispersing polyelectrolyte can have, for instance, a charge identical to that of the polymer synthesized, in other words, for the synthesis of cationic polyelectrolytes, a cationic dispersing polyelectrolyte can be used.

Among the dispersants that may be used as disclosed herein, non-limiting mention may be made of the cationic polyelectrolytes obtained by polymerization of 50 mol % to 100 mol % of at least one cationic monomer chosen from the salts, such as the hydrochlorides or sulfates, of dimethylaminoethyl (meth)acrylate, N-dimethylaminopropyl(meth)-acrylamide, di(meth)allylamine, (meth)acryloyloxy-ethyltrimethylammonium chloride, (meth)acrylamido-propyltrimethylammonium chloride and dimethyldiallyl-ammonium chloride, and of 50 mol % to 0 mol % of acrylamide. A polyamine such as a polyalkyleneamine may also be used.

The dispersant can be used, for example, in an amount ranging from 1% to 10% by weight, relative to the total weight of the monomers to be polymerized.

The saline aqueous solution that serves as synthesis and dispersion medium for the high molecular weight water-soluble cationic polymer is a solution in water of at least one mineral salt, for instance, those chosen from divalent anionic salts. Non-limiting examples of anionic salts that may be mentioned include ammonium sulphate, ammonium hydrogen sulphate, sodium sulphate, sodium hydrogen sulphate, magnesium sulphate, magnesium hydrogen sulphate, aluminium sulphate and aluminium hydrogen sulphate. In one embodiment of the present disclosure, the anionic salts are chosen from ammonium sulphate and sodium sulphate.

The concentration of the at least one salt should be, for example, sufficient to induce the precipitation of the high molecular weight water-soluble cationic polymer formed in the polymerization medium, and may be up to the saturation concentration of each salt. To obtain such a precipitation, the salt concentration can range, for example, from 10% to 50% by weight, relative to the total weight of the polymer solution or dispersion, for instance, greater than 15% by weight and less than 50% by weight relative to the total weight of the polymer solution or dispersion. The saline aqueous solution may also comprise monovalent salts such as sodium chloride and ammonium chloride.

The heterogeneous free-radical polymerization in aqueous medium as described above can be accompanied by a large increase in the viscosity of the reaction medium, which may be reflected by difficulties in stirring, a lack of homogeneity of the reaction medium and an increase in the particle size of the polymer particles formed. To prevent such an increase in viscosity, it has been proposed, in European Patent Application No. EP 0 943 628, to add to the polymerization medium at least one agent for preventing the increase in viscosity of the reaction medium during polymerization. The high molecular weight water-soluble cationic polymers used as disclosed herein can be prepared, for example, in the presence of at least one agent for preventing the increase in viscosity.

The at least one agent for preventing the increase in viscosity of the reaction medium can be chosen, for example, from:
(1) polycarboxylic acids and salts thereof,
(2) polyphenols,
(3) cyclic compounds comprising a hydroxyl group and a carboxyl group, or salts thereof,
(4) gluconic acid or salts thereof,
(5) the reaction products obtained by reacting a methoxyhydroquinone and/or a cationic (meth)acrylic monomer with a free-radical-generating compound, under an oxidizing atmosphere,
(6) the reaction products obtained by reacting a cationic (meth)acrylic polymer with a free-radical-generating compound, under an oxidizing atmosphere,
(7) the reaction products obtained by reacting a cationic (meth)acrylic polymer with an oxidizing agent, and mixtures thereof.

The addition of at least one agent for preventing the increase in viscosity as described above makes it possible to perform the polymerization of the water-soluble monomers described above with a low-power stirrer while at the same time avoiding the formation of coarse particles. The agents for preventing an increase in viscosity can be, for example, soluble in the aqueous reaction medium.

Non-limiting examples of (1) polycarboxylic acids and salts thereof that may be mentioned include oxalic acid, adipic acid, tartaric acid, malic acid and phthalic acid, and the salts thereof.

Non-limiting examples of (2) polyphenols that may be mentioned, for instance, include resorcinol and pyrogallol.

Non-limiting examples of (3) cyclic compounds comprising a hydroxyl group and a carboxyl group, or salts thereof that may be mentioned include m-hydroxybenzoic acid, p-hydroxybenzoic acid, salicylic acid, gallic acid and tannic acid, and the salts of these acids.

Non-limiting examples of (4) gluconic acid or salts thereof that may be mentioned include sodium gluconate, potassium gluconate, ammonium gluconate and various amine salts of gluconic acid.

Non-limiting examples of (5) the reaction products obtained by reacting a methoxyhydroquinone and/or a cationic (meth)acrylic monomer with a free-radical-generating compound, under an oxidizing atmosphere that may be mentioned include those obtained by reacting a free-radical-generating compound, under a stream of oxygenated gas, in a solution containing methoxyhydroquinone and/or a cationic (meth)acrylic monomer. The free-radical-generating compound may be an initiator commonly used for free-radical polymerization. Non-limiting examples that may be mentioned include water-soluble azo initiators such as 2,2′-azobis(2-amidinopropane) hydrochloride sold, for example, under the name V-50 by the company Wako Chemical Industries, or 2,2′-azobis[2-(2-imidazolin-2-yl)propane] hydrochloride sold, for example, under the trade name VA-044 by the company Wako Chemical Industries, or an initiator from the group of water-soluble redox agents, such as the ammonium persulphate/sodium hydrogen sulphite combination.

The agents for preventing an increase in viscosity such as (6) the reaction products obtained by reacting a cationic (meth)acrylic polymer with a free-radical-generating compound, under an oxidizing atmosphere, may be obtained by reacting a free-radical initiator, under a oxygenated atmosphere, with a dispersant as disclosed herein. The polymerization initiator may be a water-soluble azo initiator or a water-soluble redox agent as described above.

The reaction products (7) obtained by reacting a cationic (meth)acrylic polymer with an oxidizing agent may be obtained in the form of oxidized polymers of low molecular mass by oxidation of a cationic dispersant as disclosed herein obtained by polymerization of a cationic (meth)acrylic monomer, using hydrogen peroxide or a halogen as oxidizing agent.

Among the cationic (meth)acrylic monomers used for the preparation of agents for preventing an increase in viscosity of types (5), (6), and (7) non-limiting examples that may be mentioned include dimethylaminoethyl (meth)acrylate hydrochloride or sulphate, (meth)acryloyloxyethyl-trimethylammonium chloride, (meth)acryloyloxyethyl-dimethylbenzylammonium chloride, the hydrochloride or sulphate derived from N-dimethylaminopropyl(meth)-acrylamide, (meth)acrylamidopropyltrimethylammonium chloride, dimethylaminohydroxypropyl (meth)acrylate chloride or sulphate, (meth)acryloyloxyhydroxypropyl-trimethylammonium chloride and (meth)acryloyloxy-hydroxypropyldimethylbenzylammonium chloride.

The agents for preventing an increase in viscosity as set forth above may be used alone or as a mixture, in an amount ranging, for example, from 10 ppm to 10,000 ppm relative to the total weight of the reaction solution.

The water-soluble monomers polymerized by heterogeneous free-radical polymerization to obtain high molecular weight water-soluble cationic polymers are monomers comprising at least one ethylenic double bond, for example a vinyl, acrylic or allylic double bond. They may be cationic or nonionic and may be used as a mixture.

Non-limiting examples of water-soluble nonionic monomers that may be mentioned include acrylamide, methacrylamide, N-vinylformamide, N-vinylacetonamide, hydroxypropyl acrylate and hydroxypropyl methacrylate.

The at least one water-soluble cationic monomer can be chosen, for example, from di(C₁₋₄ alkyl)diallylammonium salts and the compounds of formula (I) 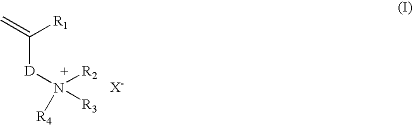
wherein:
R₁ is chosen from a hydrogen atom and methyl groups,
R₂ and R₃, which may be identical or different, are chosen from hydrogen atoms and linear and branched C₁₋₄ alkyl groups,
R₄ is chosen from a hydrogen atom, linear and branched C₁₋₄ alkyl groups, and aryl groups,
D is chosen from units of the following formula: 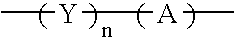
   in which Y is chosen from amide (—CO—NH—), ester (—O—CO— or —CO—O—), urethane (—O—CO—NH—), and urea (—NH—CO—NH—) functional groups,
A is chosen from linear, branched and cyclic C₁₋₁₀ alkylene groups, which may be substituted or interrupted with a divalent aromatic or heteroaromatic ring, or which may be interrupted with a hetero atom chosen from O, N, S and P, and which may comprise a ketone, amide, ester, urethane or urea functional group,
n is equal to 0 or 1, and
X⁻ is an anionic counterion such as a chloride or sulphate ion.

Non-limiting examples of water-soluble cationic monomers that may be mentioned include dimethylaminoethyl (meth)acrylate hydrochloride or sulphate, (meth)acryloyloxyethyltrimethylammonium chloride, (meth)acryloyloxyethyldimethylbenzylammonium chloride, N-dimethylaminopropyl(meth)acrylamide hydrochloride or sulphate, (meth)acrylamidopropyltrimethylammonium chloride, (meth)acrylamidopropyldimethylbenzylammonium chloride, dimethylaminohydroxypropyl (meth)acrylate hydrochloride or sulphate, (meth)acryloyloxyhydroxy-propyltrimethylammonium chloride, (meth)acryloyloxy-hydroxypropyidimethylbenzylammonium chloride and dimethyidiallylammonium chloride.

In one embodiment of the present disclosure, for example, the high molecular weight water-soluble cationic polymer is obtained by heterogeneous free-radical polymerization of a monomer mixture comprising from 0 mol % to 95.5 mol % of acrylamide and from 4.5 mol % to 100 mol % of at least one cationic monomer of formula (I).

According to another embodiment of the present disclosure, for instance, the high molecular weight water-soluble cationic polymers are obtained by polymerization of a monomer mixture comprising acrylamide and a cationic monomer of formula (I), in which the number of moles of cationic monomer of formula (I) is greater than the number of moles of acrylamide.

Among the high molecular weight water-soluble cationic polymers that may be used as disclosed herein, for example, non-limiting mention may be made of those polymerized using monomer mixtures comprising:
1) 10 mol % of acryloyloxyethyldimethylbenzyl-ammonium chloride and 90 mol % of acrylamide;
2) 30 mol % of acryloyloxytrimethylammonium chloride, 50 mol % of acryloyloxyethyldimethylbenzyl-ammonium chloride and 20 mol % of acrylamide;
3) 10 mol % of acryloyloxyethyltrimethylammonium chloride and 90 mol % of acrylamide; and
4) 30 mol % of diallyldimethylammonium chloride and 70 mol % of acrylamide.

The high molecular weight water-soluble cationic polymers used as disclosed herein have a weight-average molecular mass of greater than 1,000,000, for example, ranging from 1,000,000 to 50,000,000, such as ranging from 1,000,000 to 20,000,000. This weight-average molecular mass is determined via the RSV (Reduced Specific Viscosity) method as defined in Principles of Polymer Chemistry, Cornell University Press, Ithaca, N.Y., 1953, Chapter VII: “Determination of Molecular Weight,” pages 266-316.

The concentration of the high molecular weight water-soluble cationic polymer dispersion or solution can be, for example, chosen such that the concentration of the water-soluble polymer ranges from 0.01% to 10% by weight, and for instance, from 0.05% to 5% by weight, relative to the total weight of the final composition.

The concentration of particles of the at least one water-soluble cationic polymer as a dispersion in a saline aqueous solution in (a1) can range from 0.01% to 20% by weight, relative to the total weight of the dispersion.

The concentration of the at least one water-soluble polymer as a solution in (a2) can range from 0.01% to 20% by weight, relative to the total weight of the solution.

The at least one drawing polymer (polymer with a drawing power of greater than 5 cm) as disclosed herein can be present in an amount, for example, ranging from 0.01% to 10% by weight, such as from 0.05% to 5% by weight, relative to the total weight of the final composition.

The at least one detergent surfactant is chosen from anionic, amphoteric, nonionic and zwitterionic surfactants.

Thus, according to the present disclosure, the at least one detergent surfactant can be present in an amount ranging from 4% to 50% by weight, such as from 6% to 30% by weight, and for instance, from 8% to 25% by weight, relative to the total weight of the final composition.

Non-limiting examples of the surfactants that are suitable for carrying out the present disclosure include, for example, the following:

(i) Anionic Surfactant(s):

In the context of the present disclosure, the nature of the anionic surfactants is not a critical feature.

Thus, as non-limiting examples of anionic surfactants that may be used, alone or as mixtures, in the context of the present disclosure, mention may be made for example, of salts (for instance, alkaline salts, such as sodium salts, ammonium salts, amine salts, amino alcohol salts or magnesium salts) of the following compounds: alkyl sulphates, alkyl ether sulphates, alkylamido ether sulphates, alkylaryl polyether sulphates, monoglyceride sulphates, alkyl sulphonates, alkyl phosphates, alkylamide sulphonates, alkylaryl sulphonates, α-olefin sulphonates, paraffin sulphonates, alkyl sulphosuccinates, alkyl ether sulphosuccinates, alkylamide sulphosuccinates, alkyl sulphosuccinamates, alkyl sulphoacetates, alkyl ether phosphates, acyl sarcosinates, acyl isethionates and N-acyltaurates, the alkyl or acyl radical of all of these various compounds comprising, for example, from 12 to 20 carbon atoms. In one embodiment of the present disclosure, the aryl radical is chosen from phenyl and benzyl groups. Among the anionic surfactants that may also be used, non-limiting mention may also be made of fatty acid salts such as the salts of oleic, ricinoleic, palmitic and stearic acids, coconut oil acid or hydrogenated coconut oil acid; acyl lactylates in which the acyl radical comprises from 8 to 20 carbon atoms. Use may also be made of weakly anionic surfactants, such as alkyl-D-galactosiduronic acids and their salts, and also polyoxyalkylenated carboxylic ether acids and their salts, for instance those comprising from 2 to 50 ethylene oxide groups, and mixtures thereof. Anionic surfactants of the polyoxyalkylenated carboxylic ether acid or salt type may be, for example, chosen from those of formula (1):
R₁—(OC₂H₄)ₙ—OCH₂COOA (1)
wherein:
R₁ is chosen from alkyl, alkylamido and alkaryl groups, and n is an integer or decimal number (average value) ranging from 2 to 24, such as from 3 to 10, the alkyl radical comprising from 6 to 20 carbon atoms. For example, the aryl can be a phenyl group.

A is chosen from a hydrogen atom, ammonium, Na ions, K ions, Li ions, Mg ions, monoethanolamine residues and triethanolamine residues. Mixtures of compounds of formula (1) can also be used, for instance, mixtures in which the groups R₁ are different.

Compounds of formula (1) are sold, for example, by the company Chem Y under the name Akypo (NP40, NP70, OP40, OP80, RLM25, RLM38, RLMQ 38 NV, RLM 45, RLM 45 NV, RLM 100, RLM 100 NV, RO 20, RO 90, RCS 60, RS 60, RS 100, RO 50) or by the company Sandoz under the name Sandopan (DTC Acid, DTC).

(ii) Nonionic Surfactant(s):

Nonionic surfactants are likewise compounds that are well known per se (see, for example in this respect, Handbook of Surfactants by M. R. Porter, published by Blackie & Son (Glasgow and London), 1991, pp. 116-178) and, in the context of the present disclosure, their nature is not a critical feature. Thus, by way of non-limiting example, they can be chosen from polyethoxylated, polypropoxylated and polyglycerolated fatty alcohols; polyethoxylated, polypropoxylated and polyglycerolated fatty a-diols; polyethoxylated, polypropoxylated and polyglycerolated fatty alkylphenols; and polyethoxylated, polypropoxylated and polyglycerolated fatty acids, all having a fatty chain comprising, for example, 8 to 18 carbon atoms, it being possible for the number of ethylene oxide or propylene oxide groups to range for example, from 2 to 50, and for the number of glycerol groups to range, for instance, from 2 to 30.

Non-limiting mention may also be made of copolymers of ethylene oxide and of propylene oxide, condensates of ethylene oxide and of propylene oxide with fatty alcohols; polyethoxylated fatty amides having, for example, from 2 to 30 mol of ethylene oxide; polyglycerolated fatty amides comprising, on average, 1 to 5, such as 1.5 to 4, glycerol groups; oxyethylenated fatty acid esters of sorbitan comprising from 2 to 30 mol of ethylene oxide; fatty acid esters of sucrose; fatty acid esters of polyethylene glycol, alkylpolyglycosides, N-alkylglucamine derivatives, amine oxides such as (C₁₀-C₁₄)alkylamine oxides or N-acylaminopropylmorpholine oxides. In one embodiment of the present disclosure, the nonionic surfactants are chosen from alkylpolyglycosides

(iii) Amphoteric or Zwitterionic Surfactant(s):

Non-limiting examples of the amphoteric or zwitterionic surfactants, whose nature is not a critical feature in the context of the present disclosure, include: aliphatic secondary or tertiary amine derivatives in which the aliphatic radical is chosen from linear and branched chains comprising from 8 to 18 carbon atoms and comprising at least one water-soluble anionic group (for example carboxylate, sulphonate, sulphate, phosphate or phosphonate); non-limiting mention may also be made of (C₈-C₂₀)alkylbetaines, sulphobetaines, (C₈-C₂₀)alkylamido(C₁-C₆)alkylbetaines and (C₈-C₂₀)alkylamido(C₁-C₆)alkylsulphobetaines.

Among the amine derivatives, non-limiting mention may be made of the products sold under the name MIRANOL, as described in U.S. Pat. Nos. 2,528,378 and 2,781,354 and classified in the CTFA dictionary, 3rd edition, 1982, under the names Amphocarboxyglycinates and Amphocarboxypropionates, with the respective structures of formulae (2) and (3):
R₂—CONHCH₂CH₂—N⁺(R₃)(R₄)(CH₂COO⁻) (2)
wherein:
R₂ is chosen from alkyl radicals of an acid R₂—COOH present in hydrolyzed coconut oil, and heptyl, nonyl and undecyl radicals;
R₃ is a β-hydroxyethyl group; and
R₄ is a carboxymethyl group;
and
   R₂′—CONHCH₂CH₂—N(B)(C) (3)
   wherein:
B is chosen from —CH₂CH₂OX′ radicals,
C is chosen from —(CH₂)_{z}—Y′ radicals, with z equal to 1 or 2,
X′ is chosen from —CH₂CH₂—COOH and a hydrogen atom,
Y′ is chosen from —COOH and —CH₂—CHOH—SO₃H radicals,
R₂′ is chosen from alkyl radicals of an acid R₂′—COOH present in coconut oil or in hydrolyzed linseed oil, alkyl radicals, such as C₇, C₉, C₁₁ and C₁₃ alkyl radicals, C₁₇ alkyl radicals and their iso form, and unsaturated C₁₇ radicals.

For example, non-limiting mention may be made of the cocoamphocarboxyglycinate sold under the trade name Miranol C2M concentrate by the company Miranol.

Mixtures of surfactants, for example, mixtures of anionic surfactants, mixtures of anionic surfactants and of amphoteric, cationic or nonionic surfactants, or mixtures of cationic surfactants with nonionic or amphoteric surfactants, may be used in the compositions in accordance with the present disclosure. In one embodiment of the present disclosure, the surfactants are present as a mixture comprising at least one anionic surfactant and at least one amphoteric surfactant.

The at least one anionic surfactant can be present in an amount ranging from 4% to 50% by weight, relative to the total weight of the cosmetic composition, for example, ranging from 5% to 35% by weight, such as from 8% to 25% by weight.

The at least one amphoteric and/or nonionic surfactant, when they are present, can be present in an amount ranging, for example, from 0.5% to 20% by weight, such as from 1% to 15% by weight, relative to the total weight of the composition.

As used herein, the term “conditioner” means any agent whose function is to improve the cosmetic properties of keratin materials such as the hair, for example, the softness, disentangling, feel, smoothness and static electricity.

As used herein, the term “nonsilicone” is understood to mean the absence of silicone atom from the structure.

The at least one water-insoluble nonsilicone conditioner can be chosen from, for example, synthetic oils such as polyolefins, mineral oils, plant oils, fluoro oils or perfluoro oils, natural or synthetic waxes, compounds of ceramide type, carboxylic acid esters and also mixtures of these various compounds.

The at least one insoluble nonsilicone conditioner in accordance with the present disclosure may be solid, liquid or pasty at room temperature and at atmospheric pressure, and may be, for instance, in the form of oils, waxes, resins or gums.

The at least one insoluble conditioner can be, for example, dispersed in the compositions in the form of particles having a number-average size ranging from 2 nanometers to 100 microns, for instance, ranging from 30 nanometers to 20 microns (measured with a granulometer).

The at least one water-insoluble nonsilicone conditioner can be insoluble in water in an amount greater than or equal to 0.1% by weight in water at 25° C., i.e., it does not form a macroscopically isotropic transparent solution under these conditions.

In one embodiment, the synthetic oils can be chosen from polyolefins, such as poly-α-olefins. For instance, the poly-α-olefins may be of hydrogenated or non-hydrogenated polybutene type, such as hydrogenated or non-hydrogenated polyisobutene.

For example, isobutylene oligomers with a molecular weight of less than 1,000 and mixtures thereof with polyisobutylenes with a molecular weight of greater than 1,000, for example, ranging from 1,000 to 15,000 may be used.

Among the poly-x-olefins which can be used as disclosed herein, non-limiting mention may be made for example, of the polyisobutenes sold under the name Permethyl® 99 A, 101 A, 102 A, 104 A (n=16) and 106 A (n=38) by the company Presperse Inc., or alternatively the products sold under the name Arlamol® HD (n=3) by the company ICI (n denoting the degree of polymerization),

The poly-α-olefins may also be of of hydrogenated or non-hydrogenated polydecene type. Such products are sold, for example, under the names Ethylflo® by the company Ethyl Corp., and Arlamol® PAO by the company ICI.

The animal or plant oils can be chosen from, for instance, oils such as sweet almond oil, avocado oil, castor oil, olive oil, jojoba oil, liquid jojoba wax, sunflower oil, wheat germ oil, sesame oil, groundnut oil, grapeseed oil, soybean oil, rapeseed oil, safflower oil, coprah oil, corn oil, hazelnut oil, karite butter, palm oil, apricot kernel oil, calophyllum oil and mixtures thereof.

It is also possible to use natural or synthetic essential oils such as, for example, eucalyptus oil, lavendin oil, lavender oil, vetiver oil, Litsea cubeba oil, lemon oil, sandalwood oil, rosemary oil, camomile oil, savory oil, nutmeg oil, cinnamon oil, hyssop oil, caraway oil, orange oil, geraniol oil, cade oil and bergamot oil.

The composition according to the present disclosure may further comprise at least one wax chosen from animal, plant, and mineral waxes.

As used herein, the term “wax” is understood to mean a lipophilic compound that is solid at room temperature(25° C.), with a reversible solid/liquid change of state, having a melting point of greater than 40° C. and which may be as high as 200° C., and exhibiting an anisotropic crystalline organization in the solid state. In general, the size of the wax crystals is such that the crystals diffract and/or scatter light, conferring a more or less opaque cloudy appearance on the composition comprising them. By heating the wax to its melting point, it is possible to make it miscible with oils and to form a microscopically homogeneous mixture, but upon bringing the temperature of the mixture back to room temperature, recrystallization of the wax from the oils in the mixture is obtained which can be detected microscopically and macroscopically (opalescence).

Among waxes which can be used according to the present disclosure, non-limiting mention may be made of waxes of animal origin such as beeswax, spermaceti, lanolin wax and lanolin derivatives; plant waxes such as carnauba wax, candelilla wax, Ouricury wax, Japan wax, cocoa butter, cork fiber or sugar cane waxes; mineral waxes, for example paraffin wax, petroleum jelly wax or lignite wax, or microcrystalline waxes, ozokerite, olive tree wax, rice wax, hydrogenated jojoba wax or the absolute waxes of flowers such as the essential wax of blackcurrant flower sold by the company Bertin (France), animal waxes such as beeswaxes, or modified beeswaxes (cerabellina); other waxes or waxy starting materials which can be used according to the present disclosure include, for example, marine waxes such as the product sold by the company Sophim under the reference M82, and polyethylene waxes or polyolefins in general, and mixtures thereof.

For a further definition of waxes, non-limiting mention may be made, for example, of P. D. Dorgan, Drug and Cosmetic Industry, December 1983, pp. 30-33.

In one aspect of the present disclosure, the at least one wax may be chosen from, for example, carnauba wax, candelilla wax, alfalfa wax, paraffin wax, ozokerite, plant waxes such as olive tree wax, rice wax, hydrogenated jojoba wax or the absolute waxes of flowers such as the essential wax of blackcurrant flower sold by the company Bertin (France), animal waxes such as beeswaxes, or modified beeswaxes (cerabellina); marine waxes such as the product sold by the company Sophim under the reference M82, and polyethylene waxes or polyolefins in general.

According to the present disclosure, the compounds of ceramide type can be, for example, natural or synthetic ceramides and/or glycoceramides and/or pseudoceramides and/or neoceramides.

Compounds of ceramide type are described, for example, in German Patent Application Nos. DE 4 424 530, DE 4 424 533, DE 4 402 929, and DE 4 420 736; International Patent Application Nos. WO 95/23807, WO 94/07844, WO 95/16665, WO 94/07844, WO 94/24097 and WO 94/10131, French Patent Application No. FR-2 673 179, and European Patent Application Nos. EP-A-0 227 994 and EP-A-0 646 572, the teachings of which are included herein by way of reference.

Among the compounds of ceramide type that may be used according to the present disclosure, non-limiting mention may be made, for example of:
2-N-linoleoylaminooctadecane-1,3-diol,
2-N-oleoylaminooctadecane-1,3-diol,
2-N-palmitoylaminooctadecane-1,3-diol,
2-N-stearoylaminooctadecane-1,3-diol,
2-N-behenoylaminooctadecane-1,3-diol,
2-N-[2-hydroxypalmitoyl]aminooctadecane-1,3-diol,
2-N-stearoylaminooctadecane-1,3,4-triol, such as N-stearoylphytosphingosine,
2-N-palmitoylaminohexadecane-1,3-diol,
bis(N-hydroxyethyl-N-cetyl)malonamide,
N-(2-hydroxyethyl)-N-(3-cetyloxy-2-hydroxypropyl)cetylamide,
N-docosanoyl-N-methyl-D-glucamine,
and mixtures of these compounds.

The fluoro oils can be chosen from, for example, the perfluoropolyethers described for instance, in European Patent Application No. EP-A-486 135 and the fluorohydrocarbon compounds described for instance in International Patent Application No. WO 93/11103. The teachings of these two patent applications are included in their entirety in the present application by way of reference.

As used herein, the term “fluorohydrocarbon compounds” is understood to mean compounds whose chemical structure comprises a carbon skeleton in which certain hydrogen atoms have been replaced with fluorine atoms.

The fluoro oils can also be fluorocarbons such as fluoroamines, for example perfluorotributylamine, fluorohydrocarbons, for example perfluorodecahydronaphthalene, fluoro esters and fluoro ethers. The perfluoropolyethers are sold, for example, under the trade names Fomblin by the company Montefluos, and Krytox by the company Du Pont.

Among the fluorohydrocarbon compounds, non-limiting mention may also be made of fluorine-comprising fatty acid esters such as the products sold under the name Nofable FO by the company Nippon Oil.

The fatty alcohols may be chosen from linear or branched C₈-C₂₂ fatty alcohols; they are optionally oxyalkylenated with 1 to 15 mol of alkylene oxide or polyglycerolated with 1 to 6 mol of glycerol. For example, the alkylene oxide can be ethylene oxide and/or propylene oxide.

The carboxylic acid esters can be chosen from mono-, di-, tri- or tetracarboxylic esters. The monocarboxylic acid esters can be, for example, linear or branched, saturated or unsaturated C₁-C₂₆ aliphatic acid monoesters of linear or branched, saturated or unsaturated, C₁-C₂₆ aliphatic alcohols, the total carbon number of these esters being greater than or equal to 10.

Among the monoesters, non-limiting mention may be made of dihydroabietyl behenate; octyldodecyl behenate; isocetyl behenate; cetyl lactate; C₁₂-C₁₅ alkyl lactate; isostearyl lactate; lauryl lactate; linoleyl lactate; oleyl lactate; (iso)stearyl octanoate; isocetyl octanoate; octyl octanoate; cetyl octanoate; decyl oleate; isocetyl isostearate; isocetyl laurate; isocetyl stearate; isodecyl octanoate; isodecyl oleate; isononyl isononanoate; isostearyl palmitate; methylacetyl ricinoleate; myristyl stearate; octyl isononanoate; 2-ethylhexyl isononate; octyl palmitate; octyl pelargonate; octyl stearate; octyldodecyl erucate; oleyl erucate; ethyl and isopropyl palmitates; 2-ethylhexyl palmitate; 2-octyidecyl palmitate; alkyl myristates such as isopropyl, butyl, cetyl or 2-octyldodecyl myristate; hexyl stearate; butyl stearate; isobutyl stearate; dioctyl malate; hexyl laurate; 2-hexyldecyl laurate.

C₄-C₂₂ di- or tricarboxylic acid esters of C₁-C₂₂ alcohols and mono-, di- or tricarboxylic acid esters of C₂-C₂₆ di-, tri-, tetra- or pentahydroxy alcohols can also be used. Non-limiting mention may be made of, for example: diethyl sebacate; diisopropyl sebacate; diisopropyl adipate; di-n-propyl adipate; dioctyl adipate; diisostearyl adipate; dioctyl maleate; glyceryl undecylenate; octyidodecylstearoyl stearate; pentaerythrityl monoricinoleate; pentaerythrityl tetraisononanoate; pentaerythrityl tetrapelargonate; pentaerythrityl tetraisostearate; pentaerythrityl tetraoctanoate; propylene glycol dicaprylate/dicaprate; tridecyl erucate; triisopropyl citrate; triisostearyl citrate; glyceryl trilactate; glyceryl trioctanoate; trioctyldodecyl citrate; and trioleyl citrate.

Among the esters mentioned above, further non-limiting mention may be made of ethyl and isopropyl palmitates, 2-ethylhexyl palmitate, 2-octyldecyl palmitate, alkyl myristates such as isopropyl, butyl, cetyl or 2-octyldodecyl myristate, hexyl stearate, butyl stearate, isobutyl stearate; dioctyl malate, hexyl laurate, 2-hexyldecyl laurate, isononyl isononanoate and cetyl octanoate.

According to the present disclosure, the at least one water-insoluble nonsilicone conditioner can be present in an amount ranging from 0.001% to 20% by weight, such as from 0.01% to 10% by weight, from 0.05% to 5% by weight, and for instance, from 0.1% to 3% by weight, relative to the total weight of the final composition.

According to one embodiment of the present disclosure, the compositions may also comprise at least one additional cationic polymer other than the drawing polymer (polymer having a drawing power of greater than 5 cm) as disclosed herein.

The additional cationic polymers that may be used in accordance with the present disclosure may be chosen from all those already known per se as improving the cosmetic properties of the hair, i.e. for instance, those described in European Patent Application No. EP-A-0 337 354 and in French Patent Application Nos. FR-A-2,270,846, 2,383,660, 2,598,611, 2,470,596 and 2,519,863, and having a suitable cationic charge density.

As used herein, the term “cationic polymer” is understood to mean any polymer comprising cationic groups and/or groups that may be ionized into cationic groups.

Among the additional cationic polymers that may be used, non-limiting mention may be made of those comprising units comprising primary, secondary, tertiary and/or quaternary amine groups that either may form part of the main polymer chain or may be borne by a side substituent directly attached thereto.

The additional cationic polymers can have a number-average or weight-average molar mass ranging from 500 to 5×10⁶, for example, from 10³ to 3×10⁶.

Among the additional cationic polymers that may be used, non-limiting mention may also be made of the polymers of the polyamine, polyamino amide and polyquaternary ammonium type. These are known products.

The polymers of the polyamine, polyamino amide and polyquaternary ammonium type that may be used as disclosed herein, include, for example, those described in French Patent Nos. 2,505,348 and 2,542,997. Among these polymers, non-limiting mention may be made of:

(1) homopolymers or copolymers derived from acrylic or methacrylic esters or amides and comprising at least one of the units of the following formulae: 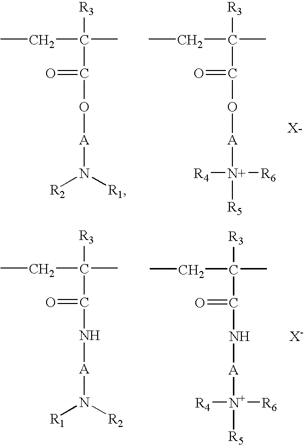
wherein:
R₃, which may be identical or different, is chosen from hydrogen atoms and CH₃ radicals;
A, which may be identical or different, is chosen from linear and branched alkyl groups of 1 to 6 carbon atoms, such as 2 to 3 carbon atoms, and hydroxyalkyl groups of 1 to 4 carbon atoms;
R₄, R₅ and R₆, which may be identical or different, are chosen from alkyl groups comprising from 1 to 18 carbon atoms and benzyl radicals; for example, alkyl groups comprising from 1 to 6 carbon atoms;
R₁ and R₂, which may be identical or different, are chosen from hydrogen atoms and alkyl groups comprising from 1 to 6 carbon atoms, such as methyl or ethyl;
X is an anion derived from a mineral or organic acid, such as a methosulphate anion or a halide such as chloride or bromide.

The copolymers of family (1) can also comprise at least one unit derived from comonomers that may be chosen from the family of acrylamides, methacrylamides, diacetone acrylamides, acrylamides and methacrylamides substituted on the nitrogen with lower (C₁-C₄) alkyls, acrylic or methacrylic acids or esters thereof, vinyllactams such as vinylpyrrolidone or vinylcaprolactam, and vinyl esters.

Thus, among these copolymers of family (1), non-limiting mention may be made of:
copolymers of acrylamide and of dimethylaminoethyl methacrylate quaternized with dimethyl sulphate or with a dimethyl halide, such as the product sold under the name Hercofloc by the company Hercules,
copolymers of acrylamide and of methacryloyloxyethyltrimethylammonium chloride described, for example, in European Patent Application No. EP-A-080 976 and sold under the name Bina Quat P 100 by the company Ciba Geigy,
copolymers of acrylamide and of methacryloyloxyethyltrimethylammonium methosulphate sold under the name Reten by the company Hercules,
quaternized or non-quaternized vinylpyrrolidone/dialkylaminoalkyl acrylate or methacrylate copolymers. These polymers are described in detail in French Patent Nos. 2,077,143 and 2,393,573,
dimethylaminoethyl methacrylate/vinylcaprolactam/vinylpyrrolidone terpolymers,
vinylpyrrolidone/methacrylamidopropyldimethylamine copolymers,
and quaternized vinylpyrrolidone/dimethylaminopropylmethacrylamide copolymers.

(2) cationic polysaccharides, for example, cationic celluloses and cationic galactomannan gums. Among the cationic polysaccharides that may be used, non-limiting mention may be made of cellulose ether derivatives comprising quaternary ammonium groups, cationic cellulose copolymers or cellulose derivatives grafted with a water-soluble quaternary ammonium monomer and cationic galactomannan gums.

The cellulose ether derivatives comprising quaternary ammonium groups are described in French Patent No. 1,492,597. These polymers are also defined in the CTFA dictionary as hydroxyethylcellulose quaternary ammoniums that have reacted with an epoxide substituted with a trimethylammonium group.

The cationic cellulose copolymers or cellulose derivatives grafted with a water-soluble quaternary ammonium monomer are described for example in U.S. Pat. No. 4,131,576, such as hydroxyalkylcelluloses, for instance hydroxymethyl-, hydroxyethyl- or hydroxypropylcelluloses grafted for example, with a methacryloyl-ethyltrimethylammonium, methacrylamidopropyltrimethylammonium or dimethyl-diallylammonium salt.

The cationic galactomannan gums are described for instance, in U.S. Pat. Nos. 3,589,578 and 4,031,307, such as guar gums containing trialkylammonium cationic groups. Use can be made, for example, of guar gums modified with a salt (e.g. chloride) of 2,3-epoxypropyltrimethylammonium.

(3) polymers comprising piperazinyl units and divalent alkylene or hydroxyalkylene radicals comprising straight or branched chains, optionally interrupted by oxygen, sulphur or nitrogen atoms or by aromatic or heterocyclic rings, and also the oxidation and/or quaternization products of these polymers. Such polymers are described, for example, in French Patent Nos. 2,162,025 and 2,280,361.

(4) water-soluble polyamino amides prepared for instance, by polycondensation of an acidic compound with a polyamine; these polyamino amides can be crosslinked with an epihalohydrin, a diepoxide, a dianhydride, an unsaturated dianhydride, a bis-unsaturated derivative, a bis-halohydrin, a bis-azetidinium, a bis-haloacyldiamine, a bis-alkyl halide or alternatively with an oligomer resulting from the reaction of a difunctional compound that is reactive with respect to a bis-halohydrin, a bis-azetidinium, a bis-haloacyldiamine, a bis-alkyl halide, an epihalohydrin, a diepoxide or a bis-unsaturated derivative; the crosslinking agent being used in an amount ranging from 0.025 to 0.35 mol per amine group of the polyamino amide; these polyamino amides can be alkylated or, if they comprise at least one tertiary amine functions, they can be quaternized. Such polymers are described, for example, in French Patent Nos. 2,252,840 and 2,368,508.

(5) polyamino amide derivatives resulting from the condensation of polyalkylene polyamines with polycarboxylic acids followed by alkylation with difunctional agents. Non-limiting mention may be made, for example, of adipic acid/dialkylamino-hydroxyalkyldialkylenetriamine polymers in which the alkyl radical comprises from 1 to 4 carbon atoms, such as methyl, ethyl or propyl radicals. Such polymers are described for instance, in French Patent No. 1,583,363. Among these derivatives, non-limiting mention may be made of, for instance, the adipic acid/dimethylamino-hydroxypropyl/diethylenetriamine polymers sold under the name “Cartaretine F, F4 or F8” by the company Sandoz.

(6) polymers obtained by reaction of a polyalkylene polyamine comprising two primary amine groups and at least one secondary amine group with a dicarboxylic acid chosen from diglycolic acid and saturated aliphatic dicarboxylic acids comprising from 3 to 8 carbon atoms. The molar ratio between the polyalkylene polyamine and the dicarboxylic acid can range from 0.8:1 to 1.4:1; the polyamino amide resulting therefrom is reacted with epichlorohydrin in a molar ratio of epichlorohydrin relative to the secondary amine group of the polyamino amide ranging from 0.5:1 to 1.8:1. Such polymers are described for example, in U.S. Pat. Nos. 3,227,615 and 2,961,347.

Polymers of this type are sold, for example, under the name “Hercosett 57” by the company Hercules Inc., in the case of the adipic acid/epoxypropyl/diethylenetriamine copolymer.

(7) cyclopolymers of alkyldiallylamine or of dialkyldiallylammonium, such as the homopolymers or copolymers comprising, as main constituent of the chain, units chosen from those of formulae (I) and/or (I′): 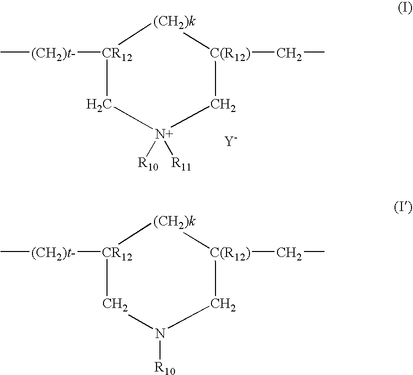
wherein k and t are equal to 0 or 1, the sum of k+t being equal to 1; R₁₂ is chosen from a hydrogen atom and methyl radicals; R₁₀ and R₁₁, which may be identical or different, are chosen from alkyl groups comprising from 1 to 6 carbon atoms, hydroxyalkyl groups in which the alkyl group comprises from 1 to 5 carbon atoms, and lower (C₁-C₄)amidoalkyl groups, or, alternatively, R₁₀ and R₁₁ can, together with the nitrogen atom to which they are attached, form heterocyclic groups such as piperidyl or morpholinyl; Y⁻ is an anion such as bromide, chloride, acetate, borate, citrate, tartrate, bisulphate, bisulphite, sulphate or phosphate. These polymers are described, for example, French Patent No. 2,080,759 and in its Certificate of Addition No. 2,190,406.

In one embodiment of the present disclosure, R₁₀ and R₁₁, independently of each other, are chosen from alkyl groups comprising from 1 to 4 carbon atoms.

Among the polymers defined above, non-limiting mention may be made of, for example, the dimethyidiallylammonium chloride homopolymer sold under the name “Merquat 100” by the company Nalco (and its homologues of low weight-average molar mass) and copolymers of diallyidimethylammonium chloride and of acrylamide.

(8) diquaternary ammonium polymers comprising repeating units of formula (II): 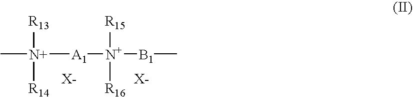
wherein:
R₁₃, R₁₄, R₁₅ and R₁₆, which may be identical or different, are chosen from aliphatic, alicyclic and arylaliphatic radicals comprising from 1 to 20 carbon atoms and lower hydroxyalkylaliphatic radicals, or alternatively R₁₃, R₁₄, R₁₅ and R₁₆, together or separately, constitute, with the nitrogen atoms to which they are attached, heterocycles optionally comprising a second hetero atom other than nitrogen, or alternatively R₁₃, R₁₄, R₁₅ and R₁₆ can be chosen from linear and branched C₁-C₆ alkyl radicals substituted with a nitrile, ester, acyl or amide group or a group —CO—O—R₁₇-D or —CO—NH—R₁₇-D where R₁₇ is an alkylene and D is a quaternary ammonium group;

A₁ and B₁ are chosen from polymethylene groups comprising from 2 to 20 carbon atoms, which groups may be linear or branched, saturated or unsaturated, and which may comprise, linked to or intercalated in the main chain, at least one aromatic ring or at least one entity chosen from oxygen and sulphur atoms, and sulphoxide, sulphone, disulphide, amino, alkylamino, hydroxyl, quaternary ammonium, ureido, amide and ester groups, and
X⁻ is an anion derived from a mineral or organic acid;
A₁, R₁₃ and R₁₅ can form, with the two nitrogen atoms to which they are attached, a piperazine ring; in addition, if A₁ is a linear or branched, saturated or unsaturated alkylene or hydroxyalkylene radical, then B₁ can also be chosen from (CH₂)ₙ—CO-D-OC—(CH₂)ₙ— radicals
   wherein D is chosen from:
a) a glycol residue of formula: —O-Z-O—, where Z is chosen from linear and branched hydrocarbon-based radicals, (CH₂—CH₂—O)ₓ—CH₂—CH₂— radicals, and —[CH₂—CH(CH₃)—O]_{y}—CH₂—CH(CH₃)— radicals
where x and y are integers ranging from 1 to 4, representing a defined and unique degree of polymerization, or any number ranging from 1 to 4 representing an average degree of polymerization;
b) a bis-secondary diamine residue such as a piperazine derivative;
c) a bis-primary diamine residue of formula: —NH—Y—NH—e, where Y is chosen from linear and branched hydrocarbon-based radicals, or alternatively divalent radical CH₂—CH₂—S—S—CH₂—CH₂—;
d) a ureylene group of formula: —NH—CO—NH—;

For example, X⁻ may be an anion such as chloride or bromide.

These polymers can have a number-average molar mass ranging from 1,000 to 100,000. Polymers of this type are described, for example, in French Patent Nos. 2,320,330, 2,270,846, 2,316,271, 2,336,434 and 2,413,907 and U.S. Pat. Nos. 2,273,780, 2,375,853, 2,388,614, 2,454,547, 3,206,462, 2,261,002, 2,271,378, 3,874,870, 4,001,432, 3,929,990, 3,966,904, 4,005,193, 4,025,617, 4,025,627, 4,025,653, 4,026,945 and 4,027,020.

It is also possible to use, for example, polymers that comprise repeating units of formula (a): 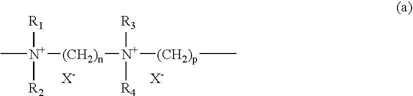
wherein R₁, R₂, R₃ and R₄, which may be identical or different, are chosen from alkyl and hydroxyalkyl radicals comprising from 1 to 4 carbon atoms, n and p are integers ranging from 2 to 20, and X⁻ is an anion derived from a mineral or organic acid. For example, in one embodiment of the present disclosure, the compound of formula (a) is the one for which R₁, R₂, R₃ and R₄ are methyl radicals, and n=3, p=6 and X═Cl, which is known as Hexadimethrine chloride according to the INCI (CTFA) nomenclature.

(9) polyquaternary ammonium polymers comprising units of formula (III): 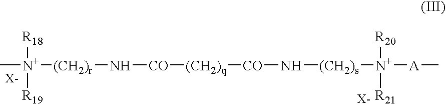
wherein:
R₁₈, R₁₉, R₂₀ and R₂₁, which may be identical or different, are chosen from hydrogen atoms, and methyl, ethyl, propyl, β-hydroxyethyl, β-hydroxypropyl and —CH₂CH₂(OCH₂CH₂)ₚOH radicals,
wherein p is equal to an integer ranging from 0 to 6, with the proviso that R₁₈, R₁₉, R₂₀ and R₂₁ are not simultaneously hydrogen atoms,
r and s, which may be identical or different, are integers ranging from 1 to 6,
q is an integer ranging from 0 to 34,
X⁻ is an anion such as a halide,
A is chosen from dihalide radicals and —CH₂—CH₂—O—CH₂—CH₂— radicals.

Such compounds are described, for example, in European Patent Application No. EP-A-122 324.

Among these products, non-limiting mention may be made, for example, of the products “Mirapol® A 15,” “Mirapol® AD1,” “Mirapol® AZ1” and “Mirapol® 175” sold by the company Miranol.

(10) quaternary polymers of vinylpyrrolidone and of vinylimidazole, for instance the products sold under the names Luviquat® FC 905, FC 550 and FC 370 by the company BASF.

(11) crosslinked polymers of methacryloyloxy(C₁-C₄)alkyltri(C₁-C₄)-alkylammonium salts, such as the polymers obtained by homopolymerization of dimethylaminoethyl methacrylate quaternized with methyl chloride, or by copolymerization of acrylamide with dimethylaminoethyl methacrylate quaternized with methyl chloride, the homo- or copolymerization being followed by crosslinking with a compound comprising olefinic unsaturation, such as methylenebisacrylamide.

Other cationic polymers that may be used as disclosed herein are cationic proteins or cationic protein hydrolysates, polyalkyleneimines, for example, polyethyleneimines, polymers comprising vinylpyridine or vinylpyridinium units, condensates of polyamines and of epichlorohydrin, polyquaternary ureylenes and chitin derivatives, such as chitosans or salts thereof; the salts that may be used include, for example, chitosan acetate, lactate, glutamate, gluconate and pyrrolidonecarboxylate.

Among these compounds, non-limiting mention may be made of chitosan with a degree of deacetylation of 90% by weight, and the chitosan pyrrolidonecarboxylate sold under the name Kytamer® PC by the company Amerchol.

Among all the cationic polymers that may be used in the context of the present invention, non-limiting mention can further be made of, for example, cationic cyclopolymers, for instance, the dimethyldiallylammonium chloride homopolymers or copolymers sold under the names “Merquat 100,” “Merquat 550” and “Merquat S” by the company Nalco, quaternary polymers of vinylpyrrolidone and of vinylimidazole, crosslinked homopolymers or copolymers of methacryloyloxy(C₁-C₄)alkyltri(C₁-C₄)alkylammonium salts, and the chitosan pyrrolidonecarboxylate sold under the name Kytamer® PC by the company Amerchol, and mixtures thereof.

The amphoteric polymers that may be used in accordance with the present disclosure can be chosen from polymers comprising units K and M randomly distributed in the polymer chain, wherein K is a unit derived from a monomer comprising at least one basic nitrogen atom and M is a unit derived from an acidic monomer comprising at least one group chosen from carboxylic and sulphonic groups, or alternatively K and M can be chosen from groups derived from zwitterionic carboxybetaine and sulphobetaine monomers;
K and M can also be chosen from cationic polymer chains comprising primary, secondary, tertiary or quaternary amine groups, in which at least one of the amine groups bears a carboxylic or sulphonic group linked via a hydrocarbon-based radical; or alternatively, K and M form part of a chain of a polymer comprising an α,β-dicarboxylic ethylene unit in which one of the carboxylic groups has been made to react with a polyamine comprising at least one amine group chosen from primary or secondary amine groups.

Among the amphoteric polymers that may be used as disclosed herein, non-limiting mention may be made of the following polymers:

(1) polymers resulting from the copolymerization of a monomer derived from a vinyl compound bearing a carboxylic group such as, for instance, acrylic acid, methacrylic acid, maleic acid, α-chloroacrylic acid, and a basic monomer derived from a substituted vinyl compound comprising at least one basic atom, such as, for example, dialkylaminoalkyl methacrylate and acrylate, dialkylaminoalkylmethacrylamide and -acrylamide. Such compounds are described in U.S. Pat. No. 3,836,537. Non-limiting mention may also be made of the sodium acrylate/acrylamidopropyltrimethylammonium chloride copolymer sold under the name Polyquart KE 3033 by the company Cognis.

The vinyl compound may also be a dialkyldiallylammonium salt such as dimethyldiallylammonium salt (for example chloride). The copolymers of acrylic acid and of the latter monomer are sold under the names Merquat 280 and Merquat 295 by the company Nalco.

(2) Polymers comprising units derived from:
a) at least one monomer chosen from acrylamides and methacrylamides substituted on the nitrogen with an alkyl radical,
b) at least one acidic comonomer comprising at least one reactive carboxylic group, and
c) at least one basic comonomer such as esters comprising primary, secondary, tertiary and quaternary amine substituents of acrylic and methacrylic acids and the product of quaternization of dimethylaminoethyl methacrylate with dimethyl or diethyl sulphate.

Among the N-substituted acrylamides or methacrylamides that may be used according to the present disclosure, non-limiting mention may be made of groups in which the alkyl radicals comprise from 2 to 12 carbon atoms, such as N-ethylacrylamide, N-tert-butylacrylamide, N-tert-octylacrylamide, N-octylacrylamide, N-decylacrylamide, N-dodecylacrylamide and the corresponding methacrylamides.

The acidic comonomers may be chosen, for example, from acrylic acid, methacrylic acid, crotonic acid, itaconic acid, maleic acid and fumaric acid and alkyl monoesters, comprising 1 to 4 carbon atoms, of maleic or fumaric acids or anhydrides.

The basic comonomers may be chosen from, for example, aminoethyl, butylaminoethyl, N,N′-dimethylaminoethyl and N-tert-butylaminoethyl methacrylates.

In one embodiment of the present disclosure, the copolymers whose CTFA (4th edition, 1991) name is Octylacrylamide/acrylates/butylaminoethyl methacrylate copolymer are used.

(3) Polyamino amides that are crosslinked and alkylated partially or totally derived from polyamino amides of formula (IV): [symbol]
CO—R₄—CO-Z [symbol] (IV)
wherein R₄ is chosen from divalent radicals derived from a saturated dicarboxylic acid; mono- and dicarboxylic aliphatic acids comprising an ethylenic double bond; esters of a lower alkanol comprising from 1 to 6 carbon atoms of these acids; and radicals derived from the addition of any one of the acids to a bis(primary) or bis(secondary) amine; and Z is chosen from bis(primary), mono- and bis(secondary) polyalkylene-polyamine radicals; for example:
a) in an amount ranging from 60 mol % to 100 mol %, the radical 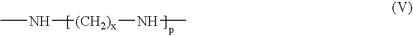
   where x is equal to 2 and p is equal to 2 or 3, or alternatively x is equal to 3 and p is equal to 2
this radical being derived from diethylenetriamine, from triethylenetetraamine or from dipropylenetriamine;
b) in an amount ranging from 0 mol % to 40 mol %, the radical (V) above in which x is equal to 2 and p is equal to 1 and which is derived from ethylenediamine, or radicals derived from piperazine: 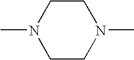
c) in an amount ranging from 0 mol % to 20 mol %, the —NH—(CH₂)₆—NH— radicals derived from hexamethylenediamine, wherein the polyamino amines are crosslinked by addition of a difunctional crosslinking agent chosen from epihalohydrins, diepoxides, dianhydrides and bis-unsaturated derivatives, using from 0.025 mol to 0.35 mol of crosslinking agent per amine group of the polyamino amide and alkylated by the action of acrylic acid, chloroacetic acid or an alkane sultone, or salts thereof.

The saturated carboxylic acids may be chosen from, for example, acids comprising 6 to 10 carbon atoms, such as adipic acid, 2,2,4-trimethyladipic acid and 2,4,4-trimethyladipic acid, terephthalic acid and acids comprising an ethylenic double bond such as, for example, acrylic acid, methacrylic acid and itaconic acid.

The alkane sultones used in the alkylation can be, for instance, propane sultone or butane sultone, and the salts of the alkylating agents can be, for example, the sodium or potassium salts.

(4) Polymers comprising zwitterionic units of formula (VI): 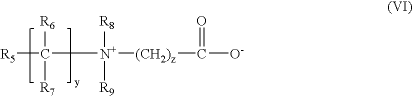
wherein R₅ is chosen from polymerizable unsaturated groups such as acrylate, methacrylate, acrylamide and methacrylamide groups; y and z are integers ranging from 1 to 3; R₆ and R₇ are chosen from hydrogen atoms, and methyl, ethyl and propyl groups, R₈ and R₉ are chosen from hydrogen atoms and alkyl radicals such that the sum of the carbon atoms in R₈ and R₉ does not exceed 10.

The polymers comprising such units can also comprise units derived from non-zwitterionic monomers such as dimethyl or diethylaminoethyl acrylate or methacrylate, or alkyl acrylates or methacrylates, acrylamides or methacrylamides, or vinyl acetate.

By way of non-limiting example, mention may be made of the copolymer of butyl methacrylate/dimethylcarboxymethylammonioethyl methacrylate.

(5) Polymers derived from chitosan comprising monomer units chosen from those of formulae (VII), (VIII) and (IX): 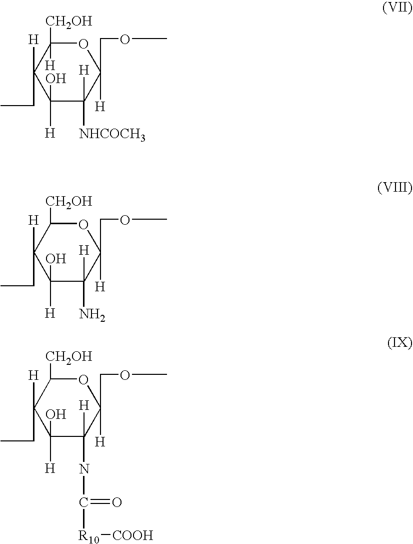
wherein the unit (VII) is present in an amount ranging from 0% to 30%, the unit (VIII) is present in an amount ranging from 5% to 50% and the unit (IX) is present in an amount ranging from 30% to 90%, it being understood that, in this unit (IX), R₁₀ is chosen from radicals of formula: 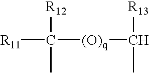
wherein
if q is equal to 0, then R₁₁, R₁₂ and R₁₃, which may be identical or different, are chosen from hydrogen atoms, methyl, hydroxyl, acetoxy and amino radicals, monoalkylamine and dialkylamine radicals that are optionally interrupted by at least one nitrogen atom and/or optionally substituted with at least one entity chosen from amine, hydroxyl, carboxyl, alkylthio and sulphonic radicals, and alkylthio radicals wherein the alkyl group bears an amino residue, and wherein at least one of the radicals R₁₁, R₁₂ and R₁₃ is, in this case, a hydrogen atom;
or, if q is equal to 1, R₁₁, R₁₂ and R₁₃ are each a hydrogen atom, and also the salts formed by these compounds with bases or acids.

(6) Polymers derived from the N-carboxyalkylation of chitosan, such as N-carboxymethylchitosan or N-carboxybutylchitosan.

(7) Polymers chosen from those of formula (X) such as those described, for example, in French Patent No. 1,400,366: 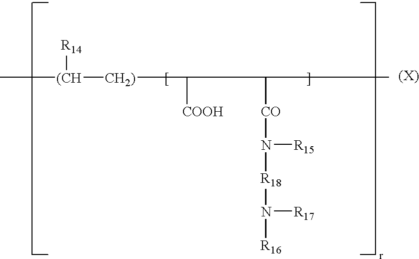
wherein R₁₄ is chosen from a hydrogen atom, CH₃O, CH₃CH₂O and phenyl radicals, R₁₅ is chosen from a hydrogen atom and lower alkyl radicals such as methyl or ethyl, R₁₆ is chosen from hydrogen atoms and lower alkyl radicals such as methyl or ethyl, R₁₇ is chosen from lower alkyl radicals such as methyl or ethyl, and —R₁₈—N(R₁₆)₂ radicals, wherein R₁₈ is chosen from —CH₂—CH₂—, —CH₂—CH₂—CH₂— and —CH₂—CH(CH₃)— radicals, and wherein R₁₆ has the meanings mentioned above, and also the higher homologues of these radicals and comprising up to 6 carbon atoms, and wherein r is an integer greater than 1.

(8) Amphoteric polymers of the type -D-X-D-X— chosen from:
a) polymers obtained by the action of chloroacetic acid or sodium chloroacetate on compounds comprising at least one unit of formula (XI):
   -D-X-D-X-D- (XI)
   where D is the radical: 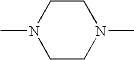
   and X is chosen from E and E′, wherein E or E′, which may be identical or different, are divalent radicals chosen from straight or branched chain alkylene radicals comprising up to 7 carbon atoms in the main chain, which can be unsubstituted or substituted with hydroxyl groups and that may also comprise at least one heteroatom chosen from oxygen, nitrogen and sulphur atoms, and 1 to 3 aromatic and/or heterocyclic rings; wherein the oxygen, nitrogen and sulphur atoms are present in the form chosen from ether, thioether, sulphoxide, sulphone, sulphonium, alkylamine, alkenylamine, hydroxyl, benzylamine, amine oxide, quaternary ammonium, amide, imide, alcohol, ester and urethane groups;
b) polymers of formula:
   -D-X-D-X— (XII)
   wherein D is the radical 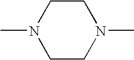
   and X is chosen from E and E′ and at least once E′; wherein E has the meaning given above and E′ is a divalent radical chosen from straight and branched chain alkylene radicals comprising up to 7 carbon atoms in the main chain, which are unsubstituted or substituted with at least one hydroxyl radical and comprising at least one nitrogen atom, wherein the nitrogen atom is substituted with an alkyl chain that is optionally interrupted by an oxygen atom and comprises at least one functional group chosen from carboxyl and hydroxyl functional groups, and betainized by reaction with chloroacetic acid or sodium chloroacetate.

(9) (C₁-C₅)alkyl vinyl ether/maleic anhydride copolymers partially modified by semiamidation with an N,N-dialkylaminoalkylamine such as N,N-dimethylaminopropylamine or by semiesterification with an N,N-dialkanolamine. These copolymers can also comprise other vinyl comonomers such as vinylcaprolactam.

In one embodiment of the present disclosure, the amphoteric polymers of family (1) are used.

According to the present disclosure, the at least one additional cationic or amphoteric polymer can be present in a total amount ranging from 0.001% to 20% by weight, for example, from 0.01% to 10% by weight, such as from 0.02% to 5% by weight, relative to the total weight of the final composition.

The additional cationic polymer can be used in a weight ratio with the high molecular weight water-soluble polymer ranging from 100 to 0.0005, for instance, from 20 to 0.01, and such as from 1 to 0.01.

According to another aspect of the present disclosure, the compositions may also comprise at least one silicone.

Among the silicones that may be used in the compositions as disclosed herein, non-limiting mention may be made, for example, of volatile or non-volatile, cyclic or acyclic, branched or unbranched, organomodified or non-organomodified silicones, as described below.

The silicones that may be used as disclosed herein may be soluble or insoluble in the composition, and for instance may be chosen from polyorganosiloxanes that are insoluble in the compositions of the present disclosure; they may be in the form of oils, waxes, resins or gums.

According to the present disclosure, all the silicones may be used in unmodified form or in the form of solutions, dispersions, emulsions, nanoemulsions or microemulsions.

The organopolysiloxanes are defined in greater detail, for example, in Walter Noll's “Chemistry and Technology of Silicones” (1968) Academic Press. They can be volatile or non-volatile.

When they are volatile, the silicones may be chosen from, for instance, those having a boiling point ranging from 60° C. to 260° C., such as from:
(i) cyclic silicones comprising from 3 to 7, such as 4 to 5 silicon atoms. These are, for example, octamethylcyclotetrasiloxane sold for instance under the name “Volatile Silicone 7207” by Union Carbide or “Silbione 70045 V 2” by Rhodia, decamethylcyclopentasiloxane sold under the name “Volatile Silicone 7158” by Union Carbide, and “Silbione 70045 V 5” by Rhodia, and mixtures thereof.

Non-limiting mention may also be made of cyclocopolymers of the dimethylsiloxane/methylalkylsiloxane type, such as “Volatile Silicone FZ 3109” sold by the company Union Carbide, having the chemical structure:

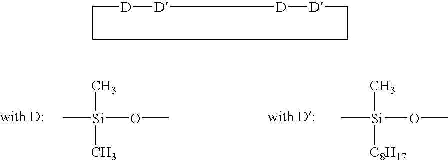

Non-limiting mention may further be made of mixtures of cyclic silicones with organosilicon compounds, such as the mixture of octamethylcyclotetrasiloxane and tetratrimethylsilylpentaerythritol (50/50) and the mixture of octamethylcyclotetrasiloxane and oxy-1,1′-bis(2,2,2′,2′,3,3′-hexatrimethylsilyloxy)neopentane;
(ii) linear volatile silicones comprising 2 to 9 silicon atoms and having a viscosity of less than or equal to 5×10⁻⁶ m²/s at 25° C. An example is decamethyltetrasiloxane sold, for instance, under the name “SH 200” by the company Toray Silicone. Silicones belonging to this category are also described in the article published in Cosmetics and Toiletries, Vol. 91, January 76, pp. 27-32, Todd & Byers “Volatile Silicone Fluids for Cosmetics.”

In one aspect of the present disclosure, non-volatile silicones are used, for instance, polyalkylsiloxanes, polyarylsiloxanes, polyalkylarylsiloxanes, silicone gums, silicone resins, and polyorganosiloxanes modified with organofunctional groups, and also mixtures thereof. These silicones may be chosen from, for example, polyalkylsiloxanes, among which non-limiting mention may be made of polydimethylsiloxanes comprising trimethylsilyl end groups and having a viscosity ranging from 5×10⁻⁶ m²/s to 2.5 m²/s at 25° C., such as ranging from 1×10⁻⁵ m²/s to 1 m²/s. The viscosity of the silicones is measured, for example, at 25° C. according to ASTM standard 445 Appendix C.

Among these polyalkylsiloxanes, mention may be made, in a non-limiting manner, of the following commercial products:
the Silbione® oils of the 47 and 70 047 series or the Mirasil® oils sold by Rhodia, such as, for example, the oil 70 047 V 500 000;
the oils of the Mirasil® series sold by the company Rhodia;
the oils of the 200 series from the company Dow Corning, such as, for instance, DC200 with a viscosity of 60,000 mm²/s;
the Viscasil® oils from General Electric and certain oils of the SF series (SF 96, SF 18) from General Electric.

Non-limiting mention may also be made of polydimethylsiloxanes comprising dimethylsilanol end groups, known under the name dimethiconol (CTFA) such as the oils of the 48 series from the company Rhodia. In this category of polyalkylsiloxanes, non-limiting mention may further be made of the products sold under the names “Abil Wax® 9800 and 9801” by the company Goldschmidt, which are poly(C₁-C₂₀)alkylsiloxanes.

The polyalkylarylsiloxanes may be chosen from, for example, polydimethyl/methylphenylsiloxanes, linear and/or branched polydimethyldiphenylsiloxanes with a viscosity ranging from 1×10⁻⁵ m²/s to 5×10⁻² m²/s at 25° C.

Among these polyalkylarylsiloxanes, non-limiting examples that may be mentioned include the products sold under the following names:
the Silbione® oils of the series 70 641 from Rhodia;
the oils of the Rhodorsil® 70 633 and 763 series from Rhodia;
the oil Dow Corning 556 Cosmetic Grade Fluid from Dow Corning;
the silicones of the PK series from Bayer, for instance the product PK20;
the silicones of the PN and PH series from Bayer, for instance the products PN1000 and PH1000;
certain oils of the SF series from General Electric, such as SF 1023, SF 1154, SF 1250 and SF 1265.

The silicone gums that may be used in accordance with the present disclosure include, for example, polydiorganosiloxanes having high number-average molecular masses ranging from 200,000 to 1,000,000, used alone or as a mixture in a solvent. This solvent can be chosen from volatile silicones, polydimethylsiloxane (PDMS) oils, polyphenylmethylsiloxane (PPMS) oils, isoparaffins, polyisobutylenes, methylene chloride, pentane, dodecane and tridecane, or mixtures thereof. Non-limiting mention may be made, for instance, of the following products:
polydimethylsiloxane,
polydimethylsiloxane/methylvinylsiloxane gums,
polydimethylsiloxane/diphenylsiloxane,
polydimethylsiloxane/phenylmethylsiloxane,
polydimethylsiloxane/diphenylsiloxane/methylvinylsiloxane.

Further non-limiting examples of products that can be used in accordance with the present disclosure are mixtures such as:
mixtures formed from a polydimethylsiloxane hydroxylated at the chain end, or dimethiconol (CTFA), and from a cyclic polydimethylsiloxane, also known as cyclomethicone (CTFA), such as the product Q2 1401 sold by the company Dow Corning;
mixtures formed from a polydimethylsiloxane gum with a cyclic silicone, such as the product SF 1214 Silicone Fluid from the company General Electric; this product is an SF 30 gum corresponding to a dimethicone, having a number-average molecular weight of 500,000, dissolved in the oil SF 1202 Silicone Fluid corresponding to decamethylcyclopentasiloxane;
mixtures of two PDMSs of different viscosities, for instance, of a PDMS gum and a PDMS oil, such as the product SF 1236 from the company General Electric. The product SF 1236 is a mixture of an SE 30 gum defined above, having a viscosity of 20 m²/s, and an SF 96 oil, with a viscosity of 5×10⁻⁶ m²/s. This product may comprise, for example, 15% SE 30 gum and 85% SF 96 oil.

The organopolysiloxane resins that can be used in accordance with the present disclosure are crosslinked siloxane systems comprising the following units:

R₂SiO_{2/2}, R₃SiO_{1/2}, RSiO_{3/2} and SiO_{4/2} wherein R is chose hydrocarbon-based groups comprising 1 to 16 carbon atoms and phenyl groups. For example, R may be chosen from C₁-C₄ lower alkyl groups, such as methyl, and phenyl groups.

Among these resins, non-limiting mention may be made of the product sold under the name “Dow Corning 593” or those sold under the names “Silicone Fluid SS 4230 and SS 4267” by the company General Electric, which are silicones of dimethyl/trimethyl siloxane structure. Non-limiting mention may also be made of the trimethyl siloxysilicate type resins sold, for example, under the names X22-4914, X21-5034 and X21-5037 by the company Shin-Etsu.

The organomodified silicones that can be used as disclosed herein are silicones as defined above and comprising in their structure at least one organofunctional groups attached via a hydrocarbon-based group.

Among the organomodified silicones, non-limiting mention may be made of polyorganosiloxanes comprising:
polyethyleneoxy and/or polypropyleneoxy groups optionally comprising C₆-C₂₄ alkyl groups, such as the products known as dimethicone copolyol sold by the company Dow Corning under the name DC 1248 or the oils Silwet® L 722, L 7500, L 77 and L 711 by the company Union Carbide, and the (C₁₂)alkylmethicone copolyol sold by the company Dow Corning under the name Q2 5200;
substituted or unsubstituted amine groups, such as the products sold under the name GP 4 Silicone Fluid and GP 7100 by the company Genesee, or the products sold under the names Q2 8220 and Dow Corning 929 or 939 by the company Dow Corning. The substituted amine groups may be, for instance, C₁-C₄ aminoalkyl groups;
thiol groups such as the products sold under the names “GP 72 A” and “GP 71” from Genesee;
alkoxylated groups such as the product sold under the name “Silicone Copolymer F-755” by SWS Silicones and Abil Wax® 2428, 2434 and 2440 by the company Goldschmidt;
hydroxylated groups such as the polyorganosiloxanes comprising a hydroxyalkyl function, described in French Patent Application No. FR-A-85/16334;
acyloxyalkyl groups such as, for example, the polyorganosiloxanes described in U.S. Pat. No. 4,957,732;
anionic groups of carboxylic type, such as, for example, in the products described in European Patent No. EP 186 507 from the company Chisso Corporation, or of alkylcarboxylic type, such as those present in the product X-22-3701E from the company Shin-Etsu; 2-hydroxyalkyl sulphonate; 2-hydroxyalkyl thiosulphate such as the products sold by the company Goldschmidt under the names “Abil® S201” and “Abil® S255;”
hydroxyacylamino groups, such as the polyorganosiloxanes described in European Patent Application No. EP 342 834. Non-limiting mention may be made, for example, of the product Q2-8413 from the company Dow Corning.

The silicones as described above may be used alone or as a mixture, in an amount ranging from 0.01% to 20% by weight, for instance ranging from 0.1% to 5% by weight, relative to the total weight of the composition.

As used herein the term “cosmetically acceptable medium” is understood to mean a medium that is compatible with keratin materials, such as the skin, the eyelashes and the hair.

The cosmetically acceptable medium may consist solely of water or comprise a mixture of water and at least one cosmetically acceptable solvent, such as a C₁-C₄ lower alcohol, for instance ethanol, isopropanol, tert-butanol or n-butanol; alkylene glycols, for instance propylene glycol, and polyol ethers; and mixtures thereof.

The composition as disclosed herein may comprise for example, from 50% to 95% by weight of water, relative to the total weight of the composition.

According to the present disclosure, the water-insoluble fatty phase can be present in a total amount ranging from 0.001% to 20% by weight, such as from 0.01% to 10% by weight, and from 0.05% to 5% by weight, relative to the total weight of the composition.

The washing compositions according to the present disclosure have a final pH ranging from 3 to 10, for example, ranging from 4.5 to 8. The pH may be adjusted to the desired value conventionally, by adding a base (organic or mineral base) to the composition, for example aqueous ammonia or a primary, secondary or tertiary (poly)amine, for instance monoethanolamine, diethanolamine, triethanolamine, isopropanolamine or 1,3-propanediamine, or alternatively by adding an acid, preferably a carboxylic acid, for instance citric acid.

The compositions as disclosed herein may further comprise viscosity regulators such as electrolytes, or thickeners (associative or non-associative thickeners). Non-limiting mention may be made of, for example, sodium chloride, sodium xylenesulphonate, scleroglucans, xanthan gums, fatty acid alkanolamides, alkyl ether carboxylic acid alkanolamides optionally oxyethylenated with up to 5 mol of ethylene oxide, such as the product sold under the name “Aminol A15” by the company Chem Y, crosslinked polyacrylic acids and acrylic acid copolymers such as crosslinked acrylic acid/C₁₀-C₃₀ alkyl acrylate copolymers. These viscosity regulators can be used in the compositions as disclosed herein in a total amount ranging from 0% to 10% by weight, relative to the total weight of the composition.

The compositions in accordance with the present disclosure may also comprise, for example, up to 5% of nacreous agents or opacifiers that are known in the art, such as, for example, fatty-chain acyl derivatives such as ethylene glycol or polyethylene glycol monostearates or distearates, and fatty-chain (C₁₀-C₃₀) ethers such as, for example, distearyl ether or 1-(hexadecyloxy)-2-octadecanol.

The compositions in accordance with the present disclosure may optionally also comprise at least one additive chosen from foam synergists such as C₁₀-C₁₈ 1,2-alkanediols or fatty alkanolamides derived from monoethanolamine or diethanolamine; silicone and non-silicone sunscreens; anionic and nonionic polymers; cationic surfactants; proteins; protein hydrolysates; fatty alcohols; linear and branched C₁₂-C₄₀ fatty acids such as 18-methyleicosanoic acid; hydroxy acids; vitamins; provitamins such as panthenol; and any other additive conventionally used in cosmetics that does not affect the properties of the compositions as disclosed herein.

Needless to say, the washing compositions according to the present disclosure may also comprise at least one adjuvant usually encountered in the field of shampoos, for instance fragrances, preserving agents, sequestering agents, softeners, dyes, moisturizers, anti-dandruff agents, anti-seborrhoeic agents, and the like.

Of course, a person skilled in the art will take care to select this or these optional additional compound(s) and/or the amounts thereof such that the beneficial properties intrinsically associated with the combination in accordance with the present disclosure are not, or are not substantially, adversely affected by the envisioned addition(s).

The compositions as disclosed herein may be in the form of optionally thickened liquids, creams or gels, for example, in a form suitable for washing, optionally caring for and/or styling the hair.

The present disclosure also relates to a cosmetic process for treating keratin materials, comprising applying an effective amount of a composition as disclosed herein to the keratin materials, and in rinsing after an optional leave-in time.

According to one embodiment of the present disclosure, the composition may be used as a shampoo.

When the compositions in accordance with the present disclosure are used as standard shampoos, they can simply be applied to wet hair and the lather generated by massaging or friction with the hands is then removed, after an optional action time, by rinsing with water, the operation possibly being repeated one or more times.

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, the numerical values set forth in the specific example are reported as precisely as possible. Any numerical value, however, inherently contain certain errors necessarily resulting from the standard deviation found in their respective testing measurements.

The following examples are intended to illustrate the invention in a non-limiting manner.

### EXAMPLES 1 to 4

Shampoo compositions in accordance with the present disclosure were prepared:

| | Constituent | | | |
|---|---|---|---|---|
| | 1 | 2 | 3 | 4 |
| Copolymer (90 mol/10 mol) of | 1% AM | — | 0.75% AM | 0.5% AM |
| acrylamide and of dimethyl- | | | | |
| aminoethyl acrylate quaternized | | | | |
| with benzyl chloride | | | | |
| (MW > 5 × 10⁶) | | | | |
| as a dispersion in a | | | | |
| concentrated saline aqueous | | | | |
| solution (Ultimer from Ondeo) | | | | |
| Copolymer (10 mol/90 mol) of | — | 0.5% AM | — | — |
| dimethylaminoethyl acrylate | | | | |
| quaternized with methyl chloride | | | | |
| and of acrylamide (MW > 5 × 10⁶) | | | | |
| as a dispersion in a | | | | |
| concentrated saline aqueous | | | | |
| solution | | | | |
| Sodium lauryl ether sulphate | 12.5% AM | 12.5% AM | 15% AM | 15% AM |
| 2.2 EO | | | | |
| Cocoamidopropylbetaine | 2.5% AM | 2.5% AM | 5% AM | 2.5% AM |
| Polyquaternium-10 | — | — | — | 0.8% |
| (Celquat SC 240 C from ICI) | | | | |
| Chitosan PCA | — | 0.3% | 0.05% | — |
| (Kytamer PC from Amerchol) | | | | |
| Isopropyl myristate | 0.5% | 1% | — | — |
| (Nikkol IPM-EX from Nikko) | | | | |
| Liquid jojoba wax | — | — | 1% | — |
| (Pure Jojoba Oil from Jojoba | | | | |
| Israel) | | | | |
| Isohexadecane | — | — | — | 1.5% |
| (Permethyl 101 A from Bayer | | | | |
| Silicones) | | | | |
| Ethanol | — | 1% | — | — |
| NaCl | — | — | — | 2% |
| Water qs | 100% | 100% | 100% | 100% |

Hair treated with these shampoos had good styling and volumizing properties.

1. A composition for washing keratin materials, comprising, in a cosmetically acceptable aqueous medium, at least one water-insoluble nonsilicone conditioner, at least one detergent surfactant chosen from anionic, nonionic, zwitterionic and amphoteric detergent surfactants, and at least one polymer with a drawing power of greater than 5 cm.

2. The composition according to claim 1, wherein the at least one detergent surfactant is chosen from anionic surfactants.

3. The composition according to claim 1, wherein the at least one detergent surfactant is present in an amount ranging from 4% to 50% by weight, relative to the total weight of the composition.

4. The composition according to claim 3, wherein the at least one detergent surfactant is present in an amount ranging from 8% to 25% by weight, relative to the total weight of the composition.

5. The composition according to claim 1, wherein the at least one polymer with a drawing power of greater than 5 cm is chosen from either:
(a1) a dispersion of particles of at least one water-soluble cationic polymer with a weight-average molecular mass of greater than 10⁶ in a saline aqueous solution, obtained by heterogeneous free-radical polymerization of water-soluble monomers with precipitation of the polymer formed; or
(a2) an aqueous saline solution of at least one water-soluble cationic polymer with a weight-average molecular mass of greater than 10⁶, obtained by heterogeneous free-radical polymerization of water-soluble monomers with precipitation of the polymer formed.

6. The composition according to claim 5, wherein the water-soluble monomers are chosen from at least one cationic monomer and/or at least one nonionic monomer comprising at least one ethylenic double bond.

7. The composition according to claim 6, wherein the at least one nonionic water-soluble monomer is chosen from acrylamide, methacrylamide, N-vinylformamide, N-vinylacetonamide, hydroxypropyl acrylate and hydroxypropyl methacrylate.

8. The composition according to claim 6, wherein the at least one cationic water-soluble monomer is chosen from di(C₁₋₄ alkyl)diallylammonium salts and the compounds of formula (I): 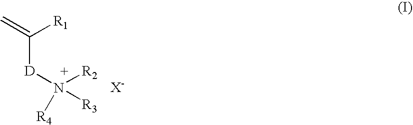
wherein:
R₁ is chosen from a hydrogen atom and methyl groups,
R₂ and R₃, which may be identical or different, are chosen from hydrogen atoms and linear and branched C₁₋₄ alkyl groups,
R₄ is chosen from a hydrogen atom, linear and branched C₁₋₄ alkyl groups, and aryl groups,
D is chosen from units of the formula: 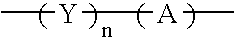
in which Y is chosen from amide, ester, urethane and urea functional groups,
A is chosen from linear, branched and cyclic C₁₋₁₀ alkylene groups, which may be substituted or interrupted with a divalent aromatic or heteroaromatic ring, or which may be interrupted with a hetero atom chosen from O, N, S and P, and which may comprise a functional group chosen from ketone, amide, ester, urethane and urea groups,
n is equal to 0 or 1, and
X⁻ is an anionic counterion.

9. The composition according to claim 8, wherein X- is chosen from halide and sulphate ions.

10. The composition according to claim 8, wherein the at least one water-soluble cationic polymer is obtained by free-radical polymerization of at least one cationic water-soluble monomer of formula (I).

11. The composition according to claim 10, wherein the at least one water-soluble cationic polymer is obtained by free-radical polymerization of a monomer mixture comprising from 0 mol % to 95.5 mol % of acrylamide and from 4.5 mol % to 100 mol % of at least one water-soluble cationic monomer of formula (I).

12. The composition according to claim 10, wherein the at least one water-soluble cationic polymer is obtained by free-radical polymerization of a monomer mixture comprising acrylamide and water-soluble cationic monomers of formula (I), wherein the number of moles of the cationic monomer of formula (I) is greater than the number of moles of acrylamide.

13. The composition according to claim 10, wherein the at least one water-soluble cationic polymer is obtained by free-radical polymerization of a monomer mixture comprising 10 mol % of acryloyloxyethyldimethylbenzylammonium chloride and 90 mol % of acrylamide.

14. The composition according to claim 10, wherein the at least one water-soluble cationic polymer is obtained by free-radical polymerization of a monomer mixture comprising 30 mol % of acryloyloxytrimethylammonium chloride, 50 mol % of acryloyloxyethyldimethylbenzylammonium chloride and 20 mol % of acrylamide.

15. The composition according to claim 10, wherein the at least one water-soluble cationic polymer is obtained by free-radical polymerization of 10 mol % of acryloyloxyethyltrimethylammonium chloride and 90 mol % of acrylamide.

16. The composition according to claim 10, wherein the at least one water-soluble cationic polymer is obtained by free-radical polymerization of 30 mol % of diallyldimethylammonium chloride and 70 mol % of acrylamide.

17. The composition according to claim 5, wherein the concentration of particles of the at least one water-soluble cationic polymer as a dispersion in a saline aqueous solution in (a1) ranges from 0.01% to 20% by weight, relative to the total weight of the dispersion.

18. The composition according to claim 5, wherein the concentration of the at least one water-soluble polymer as a solution in (a2) ranges from 0.01% to 20% by weight, relative to the total weight of the solution.

19. The composition according to claim 5, wherein the saline aqueous solution in (a1) or (a2) comprises at least one anionic salt.

20. The composition according to claim 19, wherein the at least one anionic salt is chosen from ammonium sulphate, ammonium hydrogen sulphate, sodium sulphate, sodium hydrogen sulphate, magnesium sulphate, magnesium hydrogen sulphate, aluminium sulphate and aluminium hydrogen sulphate.

21. The composition according to claim 1, wherein the at least one polymer with drawing power of greater than 5 cm is present in a total amount ranging from 0.01% to 10% by weight, relative to the total weight of the composition.

22. The composition according to claim 1, wherein the at least one water-insoluble conditioner is chosen from synthetic oils, mineral oils, plant oils, fluoro oils, perfluoro oils, natural waxes, synthetic waxes, compounds of ceramide type and carboxylic acid esters.

23. The composition according to claim 22, wherein the synthetic oils are chosen from polyolefins of hydrogenated polybutene type, non-hydrogenated polybutene type, hydrogenated polydecene type, and non-hydrogenated polydecene type.

24. The composition according to claim 22, wherein the at least one plant oil is chosen from sunflower oil, corn oil, soybean oil, avocado oil, jojoba oil, marrow oil, grapeseed oil, sesame oil, hazelnut oil, fish oils, glyceryl tricaprocaprylate, and plant and animal oils of formula R₉COOR₁₀ wherein R₉ is chosen from higher fatty acid residues comprising from 7 to 29 carbon atoms and R₁₀ is chosen from linear and branched hydrocarbon-based chains comprising from 3 to 30 carbon atoms.

25. The composition according to claim 22, wherein the at least one wax is chosen from carnauba wax, candelilla wax, alfalfa wax, paraffin wax, ozokerite, plant waxes, animal waxes, marine waxes, polyethylene and polyolefin waxes.

26. The composition according to claim 22, wherein the at least one compound of ceramide type is chosen from:
2-N-linoleoylaminooctadecane-1,3-diol,
2-N-oleoylaminooctadecane-1,3-diol,
2-N-palmitoylaminooctadecane-1,3-diol,
2-N-stearoylaminooctadecane-1,3-diol,
2-N-behenoylaminooctadecane-1,3-diol,
2-N-[2-hydroxypalmitoyl]aminooctadecane-1,3-diol,
2-N-stearoylaminooctadecane-1,3,4-triol,
2-N-palmitoylaminohexadecane-1,3-diol,
bis(N-hydroxyethyl-N-cetyl)malonamide,
N-(2-hydroxyethyl)-N-(3-cetyloxy-2-hydroxypropyl)cetylamide, and
N-docosanoyl-N-methyl-D-glucamine.

27. The composition according to claim 22, wherein the at least one carboxylic acid ester is chosen from ethyl and isopropyl palmitates, 2-ethylhexyl palmitate, 2-octyidecyl palmitate, alkyl myristates, hexyl stearate, butyl stearate, isobutyl stearate; dioctyl malate, hexyl laurate, 2-hexyldecyl laurate, isononyl isononanoate and cetyl octanoate.

28. The composition according to claim 1, further comprising at least one additional cationic polymer other than the at least one polymer with a drawing power of greater than 5 cm.

29. The composition according to claim 28, wherein the at least one additional cationic polymer is present in an amount ranging from 0.001% to 20% by weight, relative to the total weight of the final composition.

30. The composition according to claim 1, further comprising at least one silicone.

31. The composition according to claim 30, wherein the at least one silicone is present in an amount ranging from 0.01% to 20% by weight, relative to the total weight of the composition.

32. The composition according to claim 1, wherein the pH ranges from 3 to 10.

33. The process for treating hair comprising,
applying to wet hair a composition comprising, in a cosmetically acceptable aqueous medium, at least one water-insoluble nonsilicone conditioner, at least one detergent surfactant chosen from anionic, nonionic, zwitterionic and amphoteric detergent surfactants, and at least one polymer with a drawing power of greater than 5 cm,
leaving the composition on the hair for an optional action time, and then rinsing,
wherein the composition is present in an amount effective to clean and/or care for and/or condition and/or style the hair.
